(19) United States
(12) Patent Application Publication
(10) Pub. No.: US 2025/0082381 A1
(43) Pub. Date: Mar. 13, 2025

(54) CANNULATED IMPLANT REMOVAL TOOLS AND METHOD

(71) Applicant: Conventus Orthopaedics, Inc., Horsham, PA (US)

(72) Inventors: Daniel Savage, Brecksville, OH (US); Paul Hindrichs, Plymouth, MN (US); Sam Johnson, Maple Grove, MN (US); Darren DAHLIN, Excelsior, MN (US)

(21) Appl. No.: 18/884,031

(22) Filed: Sep. 12, 2024

Related U.S. Application Data
(60) Provisional application No. 63/582,200, filed on Sep. 12, 2023.

(51) Int. Cl. A61B 17/86; A61B 17/92

(52) U.S. Cl. CPC A61B 17/864; A61B 17/921

(57) ABSTRACT

Systems and methods providing for removing a cannulated implant from bone are disclosed. Systems include a trephine designed to engage and remove the shank of a cannulated screw. The trephine has an inner diameter slightly larger than the screw's shank, allowing it to advance along the shank without disturbing the bone threads. The system incorporates a K-wire, which guides the trephine during removal. The trephine's cutting edges sever bone tissue surrounding the screw shank. The method involves inserting the K-wire through the screw's cannula, aligning the trephine, and advancing it to sever the surrounding bone tissue.

### CROSS-REFERENCE TO RELATED APPLICATION

This application claims the benefit of U.S. Provisional Patent Application No. 63/582,200, which was filed on Sep. 12, 2023. This application is hereby incorporated in its entirety by reference.

### BACKGROUND

The present disclosure relates generally to surgical systems and methods, and more particularly, to systems and methods for the removal of cannulated implants, such as screws, from bone. Cannulated screws are commonly used in orthopedic procedures to secure fractured bones, stabilize bone grafts, and facilitate the healing of bone injuries. These screws are often hollow, allowing for the insertion of a guide wire, known as a K-wire, to assist with their placement and alignment during surgery.

While cannulated screws are highly effective in achieving bone fixation, complications can arise, particularly when the screw head fractures or when the screw becomes stripped or embedded in the bone. In such cases, the remaining shank of the screw must be removed to prevent further complications and to allow for the placement of a new implant. Traditional methods of removing such screws often involve drilling out the screw, which can cause significant damage to the surrounding bone and compromise the integrity of the bone threads. This damage can complicate subsequent procedures and hinder the proper seating of replacement implants.

Existing removal techniques also suffer from a lack of precision, as they often rely on reamers or other tools that may not precisely match the size of the screw shank. This can lead to the removal of more bone than necessary, further weakening the bone structure and potentially leading to additional surgical interventions. Additionally, these methods can be time-consuming and technically challenging, requiring significant surgical skill to avoid inadvertent damage to the bone.

### BRIEF SUMMARY OF THE INVENTION

To address these challenges, the present disclosure provides a system and method for the removal of cannulated screws that minimizes bone damage and preserves the integrity of the bone threads. Embodiments disclosed herein address limitations of existing screw removal techniques by providing a system and method that improves the precision, efficiency, and safety of cannulated screw removal, thereby enhancing the overall effectiveness of orthopedic surgical procedures.

The system employs a trephine, a cylindrical cutting tool designed to engage the shank of the cannulated screw with a high degree of precision. The trephine is dimensioned to advance along the shank of the screw without disturbing the surrounding bone threads, thereby preserving the bone structure for future implants.

The system further includes the use of K-wires, which are inserted through the cannula of the screw to guide the trephine during the removal process. The K-wires ensure that the trephine follows the correct trajectory, reducing the risk of deviation and minimizing damage to the surrounding bone. The trephine and K-wires can be used in conjunction with manual or powered tools, providing flexibility depending on the specific surgical requirements.

The disclosed system and method offer significant advantages over traditional screw removal techniques. By preserving the bone threads and minimizing bone damage, the system facilitates the placement of replacement implants, improving patient outcomes. Additionally, the precision provided by the trephine and K-wires reduces the technical challenges associated with screw removal, making the procedure more accessible to surgeons and reducing the risk of complications.

In some embodiments, a system for removing a cannulated implant can include a trephine designed to engage and remove the shank of a cannulated screw from a bone. The trephine can be configured with an inner diameter slightly larger than the outer diameter of the screw shank, allowing it to advance along the shank without enlarging the bone bore. The outer diameter can be smaller than the major diameter of the screw threads, enabling the trephine to cut through bone tissue surrounding the shank while preserving the bone threads. The distal end of the trephine can include cutting edges configured to sever bone tissue during the removal process. The trephine can be constructed from biocompatible materials, such as surgical-grade stainless steel or titanium, to provide durability and precision during surgery.

In some embodiments, a system for removing a cannulated implant can include both a trephine and a K-wire. The K-wire can be configured to extend through the cannula of a cannulated screw, guiding the trephine along the length of the screw shank during removal. The K-wire can have a smooth or threaded surface and can be operatively coupled with the trephine to enable simultaneous or sequential advancement through the bone tissue. For example, the K-wire can ensure accurate alignment of the trephine, minimizing bone damage and facilitating controlled removal of the screw shank and threads.

In some embodiments, a method for removing a cannulated implant can include inserting a K-wire through the cannula of a cannulated screw implanted in bone. The method can involve aligning a trephine with the K-wire, where the trephine is configured to engage and remove the screw shank. The method can include advancing the trephine along the K-wire, with the cutting edges severing the bone tissue surrounding the screw shank. The process can conclude with removing the trephine, K-wire, and cannulated screw from the bone while preserving the bone threads. For example, this method ensures that the surrounding bone remains intact, allowing for the subsequent placement of a replacement implant without the need for additional bone modification.

In some embodiments, the system can be further enhanced by integrating the trephine with a handle or powered tool. The handle can be ergonomically designed to provide the clinician with tactile feedback, allowing for precise control during the manual removal of the cannulated screw. In other variations, the system can include an attachment mechanism, such as an AO quick-connect feature, allowing the trephine to be easily attached or detached from a handle or powered surgical device, enhancing flexibility during surgery.

In some embodiments, the system can include a smooth K-wire, providing an alternative guiding method for the trephine. The smooth K-wire can be inserted through the cannula of the fractured screw, ensuring the trephine follows the correct path during removal without the need for threaded engagement. For example, the smooth K-wire can simplify the insertion and removal process, making it suitable for cases where a straightforward guiding mechanism is preferred, thereby maintaining the correct alignment of the trephine during the procedure.

### BRIEF DESCRIPTION OF THE DRAWINGS/FIGURES

FIG. 1 is an illustration of a trephine configured to engage and remove the shank of a cannulated screw from a bone, according to some embodiments.

FIG. 2 is an illustration of a removal system including a trephine and a K-wire, configured to guide the trephine during the removal of a cannulated screw, according to some embodiments.

FIG. 3 is an illustration of a removal system where the trephine is integrated with a handle, providing a manually-operated setup for removing a cannulated screw, according to some embodiments.

FIG. 4 is an illustration of a compact removal system with an integrated trephine and handle, designed for efficient manual operation, according to some embodiments.

FIG. 5 is an illustration of a removal system featuring a trephine, a smooth K-wire, and an integrated handle, configured to guide and remove a cannulated screw, according to some embodiments.

FIG. 6 is an illustration of a streamlined removal system, including a trephine and a smooth K-wire, integrated with a handle for precise manual control during the removal of a cannulated screw, according to some embodiments.

### DETAILED DESCRIPTION OF THE INVENTION

Embodiments of the present disclosure may be implemented in a variety of surgical tools and systems, including but not limited to manual, powered, or hybrid systems. These embodiments may be composed of biocompatible materials such as surgical-grade stainless steel, titanium, or other suitable alloys that ensure durability and precision during surgical procedures. Components such as trephines, K-wires, and handles may be designed to operate individually or in combination, depending on the specific requirements of the procedure.

The systems and methods described herein may be employed in various orthopedic surgical environments, including but not limited to trauma, reconstructive, or revision surgeries. The embodiments may be adapted for use with a wide range of implant types and bone structures, ensuring compatibility with existing surgical protocols and standards. Additionally, the designs may be tailored to accommodate specific patient anatomy or surgical conditions, providing flexibility in application.

In some embodiments, the surgical tools may be configured for single-use or reusable applications. Reusable components may be designed to withstand sterilization processes, including autoclaving, without degradation of performance. In contrast, single-use components may be manufactured with cost-effective materials while maintaining the necessary precision and biocompatibility required for safe and effective use.

It should be appreciated that the systems and methods described herein may be employed by trained surgical personnel and may require specific techniques or procedures to be followed. The instructions for use may include detailed steps for assembling, operating, and maintaining the surgical tools, ensuring that the procedures are carried out with the highest level of accuracy and patient safety.

The methods described in this disclosure may be implemented as part of a broader surgical procedure, where steps may be performed in a specific sequence or varied depending on the surgical context. The sequence of operations described herein is merely illustrative, and those skilled in the art will recognize that certain steps may be omitted, reordered, or supplemented with additional steps without departing from the scope of the invention.

Further, the dimensions and configurations of the surgical tools, including but not limited to the trephine, K-wire, and handle, may be customized based on the surgical application. Variations in size, shape, and material composition may be made to optimize the performance of the tools for specific bone types, implant sizes, or surgical techniques.

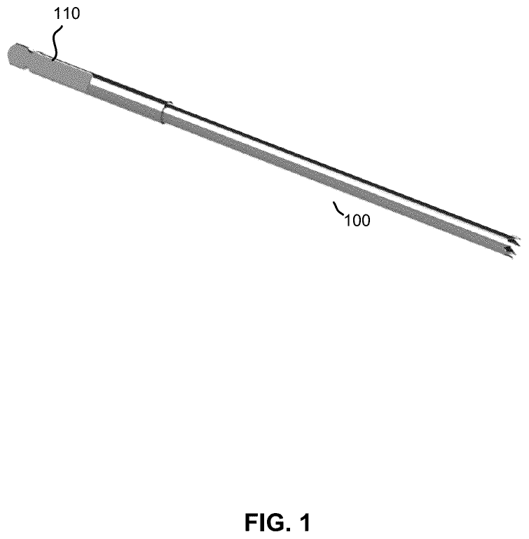

FIG. 1 illustrates an embodiment of a removal system 100 for cannulated implants, focusing specifically on the trephine 100, which is designed for the precise extraction of a cannulated bone screw with a fractured head. System 100 is configured to remove the remaining screw shank and threaded portion from the bone while preserving the surrounding bone structure, allowing for the insertion of a similarly sized implant post-removal.

The trephine 100, depicted in FIG. 1, is a cylindrical, hollow cutting tool designed to engage with the shank of the cannulated screw. The inner diameter (ID) of the trephine 100 is slightly larger than the outer diameter of the screw shank, allowing the trephine 100 to advance along the length of the screw shank without significantly enlarging the original bone bore. This precise sizing and sizing relationship can minimize bone damage and preserve the integrity of the bone for subsequent procedures.

The outer diameter of the trephine 100 is less than the major diameter of the screw threads, ensuring that the bone threads are preserved during the removal process. The distal end of the trephine 100 features cutting edges that are designed to sever the bone tissue surrounding the screw shank, allowing the trephine 100 to advance through the bone. These cutting edges may be straight or serrated, depending on the application and the density of the bone being treated.

The proximal end of the trephine 100 may be designed to interface with a handle or other surgical tool, possibly through a quick-connect feature. Trephine 100 includes quick-connect 110 that facilitates attachment of trephine 100 with another surgical tool. Trephine 100 can be configured to interface with a handle (not shown in FIG. 1). This connection allows trephine 100 to be securely attached to a handle, providing the necessary stability and control during the removal procedure.

In the surgical operation, the trephine 100 would typically be guided by a K-wire, which aligns the trephine 100 with the screw shank to ensure accurate and controlled removal. Trephine 100 is constructed from a durable, biocompatible material such as surgical-grade stainless steel or titanium. This ensures that the trephine 100 can withstand the forces required to cut through bone while maintaining the sharpness and structural integrity necessary for repeated use.

In some embodiments, a tight trephine can help to sever any ingrowth/ongrowth connection between the surrounding bone tissue and a bone screw device shank, to facilitate post-healing removal. Also, in some embodiments a castellation can be provided to the proximal portion of the implant threads which would engage with the distal end of the trephine enabling the trephine to drive the screw directly in reverse.

In some embodiments, a succession of K-wires may be used to accomplish removal. Depending on the nature of the screw fracture, different K-wire sizes can be used to achieve removal. In one non-limiting example, if a fracture occurs in a removal torque situation (counterclockwise) and grows the ID of the implant then K-wires of an appropriate size can be used to address such a fracture. In another example, if a fracture occurs in an insertion torque situation (clockwise) and the ID does not necessarily grow, then K-wires of an appropriate size can be used for operating on the fracture.

In some embodiments, the inner geometry of the trephine can be varied to help with these differences as well. For example, the trephine may have a tapered entry or a stepped counterbore. In another embodiment, the trephine has a smooth inner diameter or an inner diameter stepped from larger at the entry point smoothly down to a smaller inner diameter. In one non-limiting example, the step can be located approximately 5 mm from the distal end and/or can be moved. In another embodiment, the inner diameter can be stepped down to a smaller diameter, and then stepped back up (e.g., an hourglass shape of the inner diameter). In another embodiment, the inner diameter can be tapered gradually from a larger opening distally to a smaller diameter moving proximally. In some embodiments, the inner diameter can include threads for engaging an implant shank.

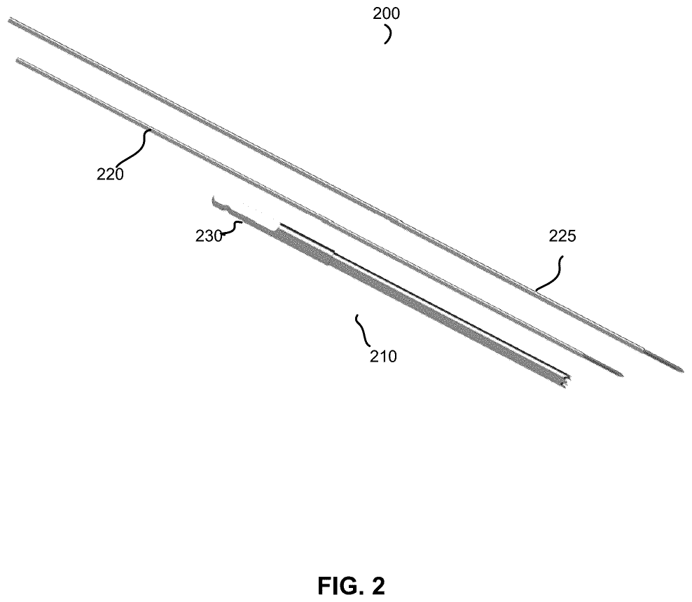

FIG. 2 illustrates a further embodiment of the removal system 200 for cannulated implants, expanding upon the system introduced in FIG. 1. This embodiment incorporates additional components and features designed to enhance the versatility and effectiveness of the system during the removal of a cannulated bone screw with a fractured head.

System 200 includes a trephine 210, similar in purpose to the trephine 100 described in FIG. 1, but with enhanced features to facilitate more complex removals. Additionally, the system includes a K-wire 220 and an attachment mechanism 230 that allows for integration with different handle types or powered devices.

The trephine 210 is a cylindrical, hollow cutting tool designed for the precise engagement and removal of a cannulated screw's shank and potentially its threaded portion from the bone. The inner diameter of the trephine 210 is designed to be fractionally larger than the outer diameter of the screw shank, allowing the trephine to advance along the shank while minimizing bone damage. The outer diameter of the trephine 210 is specifically calibrated to interact with the screw threads, ensuring that both the shank and the threaded portion of the screw can be removed if necessary.

The cutting edges at the distal end of the trephine 210 are designed to effectively sever bone tissue around the screw, facilitating its removal. These edges may be straight, serrated, or otherwise optimized depending on the bone density and specific surgical requirements.

The K-wire 220 serves as a guiding component, ensuring that the trephine 210 follows the correct path along the cannulated screw. In this embodiment, the K-wire 220 is designed to extend through the cannula of the fractured screw, providing a stable axis for the trephine 210 to follow. The K-wire 220 may include left-handed threads at its distal end, which engage with the internal diameter of the cannulated screw shank, creating an interference fit that secures the screw shank during removal.

Once the trephine 210 has advanced to the proximal aspect of the screw, the K-wire 220 may be replaced with a larger diameter K-wire 225, enhancing the interference fit and ensuring secure engagement of the screw shank and threads during the removal process.

The attachment mechanism 230 provides the means for connecting the trephine 210 to various handles or powered surgical tools. This mechanism allows the clinician to choose between manual and powered removal methods, depending on the specific needs of the procedure. The attachment mechanism 230 may include an AO quick-connect feature, which enables rapid attachment and detachment, increasing the flexibility and efficiency of the surgical process.

In the manual configuration, the attachment mechanism 230 allows the trephine 210 to be securely connected to a handle (not shown in FIG. 2), providing the surgeon with precise control over the removal process. In a powered configuration, the mechanism 230 allows the trephine 210 to be connected to a surgical drill or other powered device, enabling the application of greater torque and speed as needed.

In practice, the operation of the system 200 begins with the insertion of the K-wire 220 through the cannula of the fractured screw. The K-wire 220 serves as a guide, ensuring that the trephine 210 follows the correct trajectory along the screw shank. The trephine 210 is then advanced along the K-wire 220, cutting through the bone tissue surrounding the screw shank and threaded portion.

If necessary, the K-wire 220 can be replaced with a larger K-wire 225 to create a stronger interference fit between the screw and the trephine 210, ensuring that the entire assembly—trephine, K-wire, and screw—can be removed in a single step. The attachment mechanism 230 allows the clinician to adapt the system 200 to either manual or powered removal methods, providing flexibility based on the specific surgical scenario.

System 200 offers several advantages over simpler removal methods. The inclusion of the K-wire 220 as a guiding element ensures precise alignment and minimizes the risk of damaging surrounding bone tissue. The attachment mechanism 230 provides flexibility in choosing between manual and powered removal, making the system adaptable to various surgical environments.

The trephine 210's design, which allows for the engagement and removal of both the screw shank and its threads, ensures that the system 200 can handle a wide range of screw removal scenarios, including those involving broken or damaged screws.

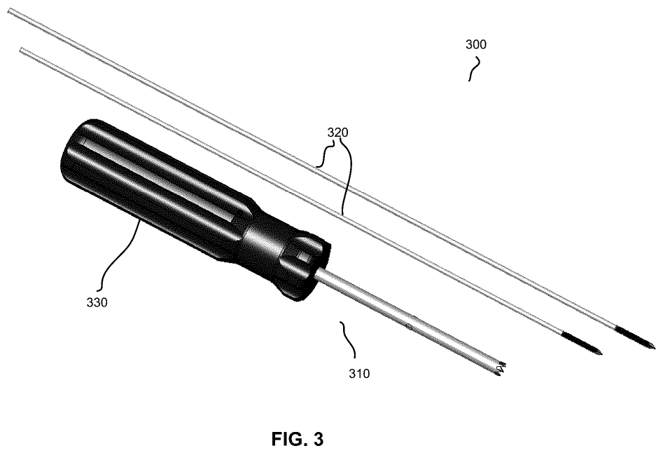

FIG. 3 illustrates an enhanced embodiment of a removal system 300 for cannulated implants, focusing on a manual setup that provides precise control during the extraction of a cannulated bone screw with a fractured head. System 300 is configured to facilitate the removal of the screw shank and its threaded portion while preserving the surrounding bone structure, making it possible to insert a similarly sized implant after the procedure.

System 300 comprises three main components: a trephine 310, K-wires 320, and a handle 330. This configuration allows for a manual, controlled removal process, providing the surgeon with tactile feedback during the procedure.

The trephine 310 is a hollow cylindrical cutting tool designed to engage with and remove the shank of a cannulated screw. The ID of the trephine 310 is slightly larger than the outer diameter of the screw shank, allowing the trephine 310 to advance along the screw shank without significantly enlarging the original bone bore. The outer diameter of the trephine 310 is smaller than the major diameter of the screw threads, which ensures that the bone threads are preserved during removal.

The cutting edges at the distal end of the trephine 310 are designed to sever the bone tissue surrounding the screw shank, allowing the trephine 310 to advance through the bone. These cutting edges may be serrated or smooth, depending on the specific application and bone density.

The K-wires 320 are long, thin wires that serve as guides for the trephine 310 during the removal process. Two K-wires are depicted in FIG. 3, each with different diameters, providing options depending on the specific conditions of the screw removal. These K-wires are inserted through the cannula of the fractured screw, extending beyond the distal end of the screw shank and into the bone. This positioning ensures that the trephine 310 follows the correct path during the removal process.

The K-wires 320 may feature left-handed threads at their distal ends, which engage with the internal diameter of the screw shank. This engagement creates an interference fit that secures the screw shank, preventing it from rotating or becoming dislodged during the removal.

The handle 330 is attached to the proximal end of the trephine 310, providing the clinician with precise manual control over the removal process. This handle is designed for ergonomic comfort, allowing the surgeon to apply the necessary force and torque while maintaining a secure grip. The handle 330 is connected to the trephine 310 in a fixed manner, ensuring that the trephine remains stable during the procedure.

In some embodiments, the handle 330 may include a quick-connect feature, allowing for easy attachment and detachment of the trephine 310. This feature provides flexibility in the surgical setting, enabling the surgeon to quickly switch between different tools as needed.

In practice, the removal process begins with the insertion of one of the K-wires 320 through the cannula of the fractured screw. The K-wire 320 serves as a guide for the trephine 310, ensuring that it follows the correct trajectory along the screw shank. The trephine 310 is then advanced along the K-wire 320, with the cutting edges severing the bone tissue surrounding the screw shank.

The handle 330 provides the surgeon with precise control over the process, allowing for careful advancement of the trephine 310 to the desired depth. Once the screw shank and its threaded portion are sufficiently engaged by the trephine 310, the entire assembly can be carefully extracted from the bone, leaving the bone threads intact.

System 300 offers significant advantages in situations where precise, controlled removal of cannulated screws is required. The manual handle 330 provides tactile feedback, allowing the surgeon to feel the resistance of the bone and adjust the force and direction as needed. The use of K-wires 320 as guides ensures accurate alignment and minimizes the risk of damaging the surrounding bone tissue.

Additionally, the ability to preserve the bone threads during removal can facilitate insertion of a similarly sized implant after the procedure, making the system 300 particularly valuable in orthopedic surgeries where maintaining bone integrity is paramount.

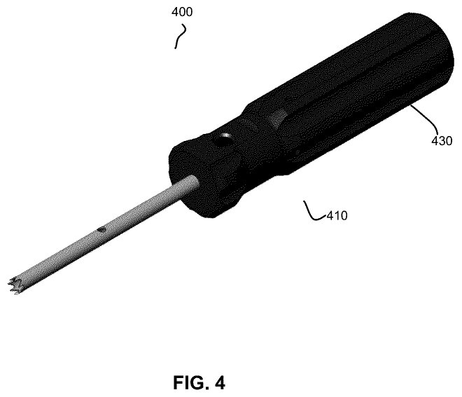

FIG. 4 illustrates an embodiment of a removal system 400 for cannulated implants, with a focus on a compact and manually-operated setup. System 400 is designed to provide efficient and controlled removal of a cannulated bone screw, particularly in situations where the screw head has fractured and separated from the shank.

System 400 includes two primary components: a trephine 410 and a handle 430. This embodiment emphasizes the integration of the trephine 410 directly with the handle 430, creating a streamlined tool that is easy to manipulate during the removal procedure.

The trephine 410 is a cylindrical, hollow cutting tool, similar in design to the trephines described in earlier embodiments. The trephine 410 features a sharp distal end with cutting edges designed to engage and remove the shank of a cannulated screw from the bone. The ID of the trephine 410 is slightly larger than the shank of the screw, allowing it to advance along the shank without damaging the surrounding bone structure.

The outer diameter of the trephine 410 is smaller than the major diameter of the screw threads, ensuring that the bone threads are preserved during the removal process. The cutting edges of the trephine 410 are designed to efficiently sever the bone tissue surrounding the screw shank, enabling the trephine to be advanced through the bone with minimal resistance.

The handle 430 is directly integrated with the trephine 410 in this embodiment, forming a single, compact tool that is easy to use in a manual removal procedure. The handle 430 is ergonomically designed to provide a comfortable grip, allowing the clinician to apply the necessary torque and pressure to advance the trephine 410 through the bone and around the screw shank.

The integration of the handle 430 with the trephine 410 simplifies the removal process by eliminating the need for multiple connections or attachments. This design is particularly advantageous in surgical settings where time and case of use are factors for consideration.

In operation, the removal process begins with the trephine 410 being positioned over the shank of the cannulated screw. The clinician then applies manual pressure and rotates the handle 430, advancing the trephine 410 through the bone tissue surrounding the screw shank. The cutting edges of the trephine 410 sever the bone tissue, allowing the trephine to advance to the desired depth.

Once the trephine 410 has fully engaged the screw shank and potentially the threaded portion, the entire tool, including the trephine 410 and handle 430, can be used to carefully extract the screw from the bone. The design of the trephine 410 ensures that the bone threads are preserved, enabling the insertion of a similarly sized implant after the removal process is complete.

System 400 integrates trephine 410 with handle 430 to create a single, compact tool that can be used effectively in a wide range of surgical scenarios. The ergonomic design of the handle 430 allows for precise control, giving the clinician tactile feedback during the removal process.

Additionally, the preservation of bone threads during the removal process is a significant benefit, as it allows for the insertion of a new implant without the need for additional bone modification. System 400 is advantageous for use in orthopedic procedures where minimizing bone damage is a priority.

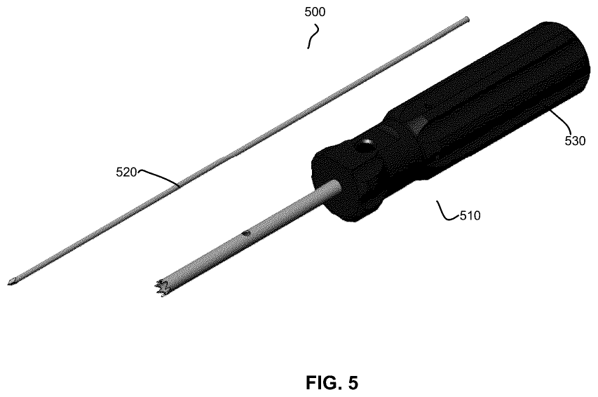

FIG. 5 illustrates another embodiment of a removal system 500 for cannulated implants, expanding upon the manual setup depicted in the previous figure. System 500 is designed to offer versatility and precision during the extraction of a cannulated bone screw, for example, when the screw head has fractured and separated from the shank.

System 500 includes trephine 510, K-wire 520, and handle 530. This embodiment builds on the previous designs by incorporating a smooth K-wire 520, offering an alternative guiding method for the trephine 510 during the removal process.

The trephine 510 is a cylindrical, hollow cutting tool, similar in design to those shown in earlier embodiments. The trephine 510 features a sharp distal end with cutting edges specifically designed to engage and remove the shank of a cannulated screw from the bone. The ID of the trephine 510 is slightly larger than the shank of the screw, allowing the trephine to advance along the shank without damaging the surrounding bone structure.

The outer diameter of the trephine 510 is smaller than the major diameter of the screw threads, ensuring that the bone threads are preserved during the removal process. The cutting edges of the trephine 510 are designed to efficiently sever the bone tissue surrounding the screw shank, enabling the trephine to be advanced through the bone with minimal resistance.

The K-wire 520 depicted in FIG. 5 is a smooth guide wire, which contrasts with the threaded K-wires shown in previous figures. This smooth K-wire 520 is inserted through the cannula of the fractured screw to guide the trephine 510 during the removal process. By providing a stable axis, the K-wire 520 ensures that the trephine 510 follows the correct path along the screw shank, reducing the risk of deviation and potential damage to the surrounding bone.

The smooth design of the K-wire 520 allows it to be easily inserted and removed without the need for threaded engagement, making it advantageous for situations where a simple guiding mechanism is preferred.

The handle 530 is directly integrated with the trephine 510, forming a single, cohesive tool that is easy to manipulate during the removal process. The handle 530 is ergonomically designed, providing a comfortable grip and allowing the clinician to apply the necessary torque and pressure to advance the trephine 510 through the bone and around the screw shank.

The integration of the handle 530 with the trephine 510 simplifies the removal process by eliminating the need for multiple connections or attachments, ensuring a streamlined and efficient procedure.

In operation, the removal process begins with the insertion of the K-wire 520 through the cannula of the fractured screw. The K-wire 520 serves as a guide for the trephine 510, ensuring that it follows the correct trajectory along the screw shank. The clinician then advances the trephine 510 along the K-wire 520, using the handle 530 to manually apply pressure and rotation.

As the trephine 510 advances, its cutting edges sever the bone tissue surrounding the screw shank, allowing the trephine to reach the desired depth. Once the trephine 510 has fully engaged the screw shank and potentially the threaded portion, the entire assembly, including the trephine 510, K-wire 520, and handle 530, can be used to carefully extract the screw from the bone.

System 500 supports a versatile approach to the removal of cannulated screws. The smooth K-wire 520 provides an alternative guiding mechanism that is easy to use and effective in maintaining the correct alignment of the trephine 510. The integration of the handle 530 with the trephine 510 ensures precise manual control, giving the clinician the ability to adjust the force and direction as needed.

Additionally, the preservation of bone threads during the removal process is a significant advantage, allowing for the insertion of a new implant without the need for additional bone modification. System 500 is advantageous for use in orthopedic procedures where minimizing bone damage and ensuring precise control are considerations.

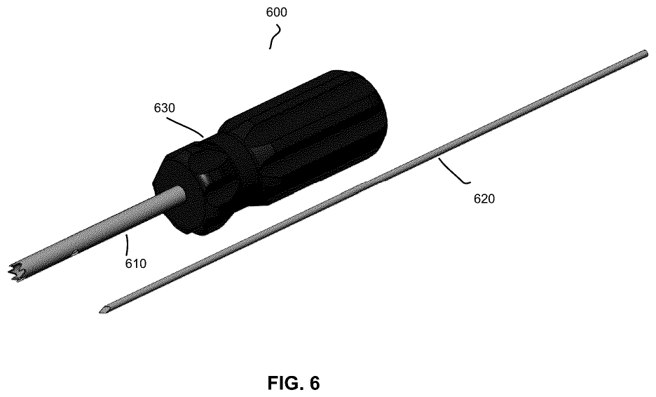

FIG. 6 illustrates another embodiment of a removal system 600 for cannulated implants, featuring a streamlined and manually-operated setup similar to those shown in previous figures. System 600 is designed to provide precise and controlled removal of a cannulated bone screw, particularly in cases where the screw head has fractured and separated from the shank.

System 600 consists of two main components: a trephine 610 and a handle 630, along with a guiding K-wire 620. This embodiment emphasizes the simplicity and efficiency of the design, focusing on a manual operation that allows for tactile feedback during the removal process.

The trephine 610 is a cylindrical, hollow cutting tool designed to engage with and remove the shank of a cannulated screw from the bone. The trephine 610 has a sharp distal end equipped with cutting edges that sever the bone tissue surrounding the screw shank. The ID of the trephine 610 is slightly larger than the shank of the screw, which allows it to advance along the screw shank without causing significant damage to the surrounding bone.

The outer diameter of the trephine 610 is smaller than the major diameter of the screw threads, ensuring that the bone threads are preserved during the removal process. The cutting edges of the trephine 610 are designed to efficiently cut through bone tissue, allowing the trephine to reach the required depth for screw removal.

The K-wire 620 serves as a guide for the trephine 610, ensuring accurate alignment during the removal process. The K-wire 620 is inserted through the cannula of the fractured screw, extending beyond the distal end of the screw shank into the bone. This positioning ensures that the trephine 610 follows a precise path during its advancement.

The K-wire 620 is depicted as a smooth wire in FIG. 6, offering a straightforward guiding mechanism without the need for threads. This design simplifies the insertion and removal of the K-wire 620, making it suitable for cases where a non-threaded guide is preferred.

The handle 630 is integrated with the trephine 610, forming a single, cohesive tool that can be easily manipulated during the removal process. The handle 630 is ergonomically designed to provide a comfortable grip, allowing the clinician to apply the necessary torque and pressure to advance the trephine 610 through the bone and around the screw shank.

This integration of the handle 630 with the trephine 610 simplifies the overall design, eliminating the need for additional connections or attachments and ensuring a more streamlined and efficient procedure.

In practice, the removal process begins with the insertion of the K-wire 620 through the cannula of the fractured screw. The K-wire 620 serves as a guide for the trephine 610, ensuring that it follows the correct trajectory along the screw shank. The clinician then advances the trephine 610 along the K-wire 620, using the handle 630 to manually control the operation.

As the trephine 610 advances, its cutting edges sever the bone tissue surrounding the screw shank, allowing the trephine to reach the desired depth. Once the trephine 610 has fully engaged the screw shank and potentially the threaded portion, the entire assembly, including the trephine 610, K-wire 620, and handle 630, can be used to carefully extract the screw from the bone.

System 600 provides smooth K-wire 620 supporting an efficient guiding mechanism that maintains the correct alignment of the trephine 610, reducing the risk of deviation and potential bone damage. The integration of the handle 630 with the trephine 610 ensures precise manual control, giving the clinician the ability to adjust the force and direction as needed.

Additionally, the preservation of bone threads during the removal process is a significant benefit, as it allows for the insertion of a new implant without requiring additional bone modification. System 600 is advantageous for use in orthopedic procedures where minimizing bone damage and ensuring precise control are considered.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A system for removing a cannulated implant, comprising:
a trephine configured to engage and remove a shank of a cannulated screw from a bone, wherein the trephine includes:
an inner diameter dimensioned to be slightly larger than an outer diameter of the shank of the cannulated screw, allowing the trephine to advance along the length of the shank;
an outer diameter that is smaller than a major diameter of threads on the cannulated screw, enabling the trephine to cut through bone tissue surrounding the shank without disturbing the bone threads;
cutting edges positioned at a distal end of the trephine (100), wherein the cutting edges are configured to sever bone tissue when implemented in a procedure to remove the shank of the cannulated screw, the bone tissue disposed around the shank of the cannulated screw, wherein the cutting edges facilitate the trephine's advancement through the bone, wherein the trephine is constructed from a biocompatible material.

2. The system of claim 1, further comprising an implement, wherein the implement is selected from a handle and a powered surgical tool, and wherein the trephine further comprises a proximal end configured to interface with the implement.

3. The system of claim 2, wherein the implement comprises an AO quick-connect feature that allows the trephine to be attached or detached from the implement.

4. The system of claim 2, wherein the implement is a handle configured to provide tactile feedback during a bone removal process, and wherein the trephine is configured to be manually operated using the handle.

5. The system of claim 1, wherein the cutting edges of the trephine are serrated.

6. The system of claim 1, wherein the trephine is constructed from surgical-grade stainless steel.

7. The system of claim 1, wherein the trephine is constructed from titanium.

8. A system for removing a cannulated implant, comprising:
a trephine configured to engage and remove a shank of a cannulated screw from a bone, wherein the trephine includes:
an inner diameter dimensioned to be slightly larger than an outer diameter of the shank of the cannulated screw, allowing the trephine to advance along the length of the shank;
an outer diameter that is smaller than a major diameter of threads on the cannulated screw, enabling the trephine to cut through bone tissue surrounding the shank without disturbing the bone threads;
cutting edges positioned at a distal end of the trephine,
wherein the cutting edges are configured to sever bone tissue disposed around the shank of the cannulated screw, thereby facilitating the trephine's advancement through the bone;
a K-wire configured to extend through a cannula of the cannulated screw, wherein the K-wire is dimensioned to guide the trephine along the length of the shank during removal, the K-wire having a smooth or threaded surface,
wherein the K-wire is inserted through the cannula of the cannulated screw and aligned with the trephine to ensure accurate and controlled removal of the shank from the bone, and
wherein the trephine and K-wire are configured to be operatively coupled to allow simultaneous or sequential advancement through the bone tissue during the removal process.

9. The system of claim 8, wherein the K-wire includes left-handed threads at a distal end configured to engage the internal diameter of the cannulated screw shank.

10. The system of claim 8, wherein the K-wire is smooth along its entire length.

11. The system of claim 9, wherein the K-wire is replaced with a larger diameter K-wire after initial advancement of the trephine to enhance the interference fit between the screw shank and the trephine.

12. The system of claim 8, wherein the K-wire and the trephine are configured to be operatively coupled via an attachment mechanism that allows for simultaneous advancement through the bone tissue.

13. The system of claim 8, wherein the K-wire includes a proximal end with an attachment feature for coupling with a handle or guiding tool.

14. The system of claim 8, wherein the trephine and K-wire are configured to be used in conjunction with a powered surgical tool.

15. A method for removing a cannulated implant from a bone, comprising:
inserting a K-wire through a cannula of a cannulated screw implanted in the bone, the K-wire being dimensioned to extend beyond the distal end of the cannulated screw into the bone;
aligning a trephine with the K-wire, the trephine configured to engage and remove a shank of the cannulated screw from the bone, the trephine comprising:
an inner diameter dimensioned to be slightly larger than an outer diameter of the shank of the cannulated screw, allowing the trephine to advance along the length of the shank;
an outer diameter that is smaller than a major diameter of threads on the cannulated screw, enabling the trephine to cut through bone tissue surrounding the shank without disturbing the bone threads;
cutting edges positioned at a distal end of the trephine, wherein the cutting edges are configured to sever bone tissue disposed around the shank of the cannulated screw, thereby facilitating the trephine's advancement through the bone;
advancing the trephine along the K-wire and through the bone tissue surrounding the shank of the cannulated screw, wherein the cutting edges of the trephine sever the bone tissue during advancement;
removing the trephine, the K-wire, and the cannulated screw from the bone, wherein the bone threads are preserved for subsequent implant insertion.

16. The method of claim 15, further comprising the step of replacing the K-wire with a larger diameter K-wire to create a stronger interference fit between the screw shank and the trephine.

17. The method of claim 15, wherein the K-wire includes left-handed threads at its distal end, and the method further comprises the step of engaging the threads with the internal diameter of the cannulated screw shank.

18. The method of claim 15, wherein the trephine is advanced along the K-wire using a manual handle configured to provide tactile feedback during a bone removal process.

19. The method of claim 15, wherein the trephine is advanced along the K-wire using a powered surgical tool.

20. The method of claim 15, wherein the cutting edges of the trephine are serrated to facilitate severing of the bone tissue surrounding the screw shank.
